(19) United States
(12) Patent Application Publication
(10) Pub. No.: US 2010/0108509 A1
(43) Pub. Date: May 6, 2010

(54) Analyte Sensor with Non-Working Electrode Layer

(75) Inventors: Kenneth M. Curry, Oceanside, CA (US); James R. Petisce, San Clemente, CA (US)

Correspondence Address: EDWARDS LIFESCIENCES CORPORATION, LEGAL DEPARTMENT, ONE EDWARDS WAY, IRVINE, CA, 92614, US

(73) Assignee: Edwards Lifesciences Corporation, Irvine, CA (US)

(21) Appl. No.: 12/609,326

(22) Filed: Oct. 30, 2009

Related U.S. Application Data
(60) Provisional application No. 61/110,219, filed on Oct. 31, 2008.

(51) Int. Cl. G01N 27/26; G01N 33/50; B05D 5/12

(52) U.S. Cl. 204/403.14; 204/400; 427/58

(57) ABSTRACT

Disclosed is an electrochemical sensors for measuring an analyte in a subject. More particularly, the sensor comprises a non-working electrode, the non-working electrode comprising a non-working electroactive surface, and a layer covering at least a portion of the non-working electroactive surface, the layer configured to prevent or reduce endogenous or exogenous components from contacting the non-working electrode surface.

### CROSS-REFERENCE TO RELATED APPLICATIONS

This application claims the benefit of U.S. Provisional Application No. 61/110,219 filed Oct. 31, 2008, entitled “Analyte Sensor with Non-Working Electrode Layer” which is hereby incorporated by reference in its entirety.

### TECHNICAL FIELD

The present disclosure relates generally to devices for measuring an analyte in a subject. More particularly, the present disclosure relates to electrochemical detection devices for measurement of an analyte that incorporates a layer on a non-working electrode, the layer reducing or eliminating changes or alterations of the electrode performance from exposure to its environment.

### BACKGROUND

In connection with in vivo measurement of analyte concentrations within body liquids, it is extremely critical that the non-working electrodes of an electrochemical sensor function to provide an accurately constant comparative potential. Inaccuracies can arise, together with consequent measuring errors, due to design and structural shortcomings, despite the thoroughly enhanced structural and design characteristics of present-day non-working electrodes.

For example, among many problems impeding the development of a practical rapid and accurate amperometric sensor is fouling of the electrodes as a result of exposure with its environment. Such events lead to decay and loss of a signal that may effect the attainment of an accurate representative of the true analyte level. This is especially problematic for continuous, in-vivo monitoring devices. Attempts to reduce fouling in amperometric sensors have been addressed in a number of ways, for example by using one or more specialty layers in a multi-membrane-based system or by incorporating labile chemical species for release into the environment about the sensor. However, these attempts have met with limited success, because performance attributes of the sensor. For example, additional layers typically reduced the detectable response and increases break-in time of the sensor. Incorporating labile anti-fouling chemicals may complicate the regulatory approval of such devices and/or potentially trigger an immune response. In certain cases, such as an intensive care units (ICUs) setting or for continuous glucose monitoring (CGM) applications, reduction or elimination of fouling of the non-working electrodes, including the reference and/or counter electrodes, would ideally be for days or perpetual. Thus, the current amperometric sensors available on the market may not be capable of achieving sustained electrode anti-fouling performance needed for some specific applications, such as ICU monitoring of analyte levels in a subject.

Generally, most commercially deployed electrochemical sensors possess an architecture having an layer on the working electrode among other layers, and only an rate-limiting diffusion layer covering the non-working electrode. Thus, in actual in-vivo use, blood components including interferants (either endogenous or exogenous) surround and come into contact with the typical sensor and its electrodes.

### SUMMARY

The embodiments herein disclosed provide a sensor comprising a non-working electrode that is suitable for use within an electrochemical sensor system for in vivo analyte concentration measurement and monitoring, such as that carried out within the bloodstream of a patient, which prevents or substantially eliminates non-working electrode fouling while providing an accurately constant comparative potential. The non-working electrode includes a layer deposited on the electroactive surface of the electrode.

It is accordingly an aspect of the present disclosure to provide a sensor comprising a non-working electrode which reduces the likelihood of having errors occur during analyte concentration measurements, especially in vivo measurements of analyte concentrations within body liquids.

In general, electrochemical analyte sensors and sensor assemblies are disclosed that provide a layer at least partially covering the non-working electrode. Such sensors provide improved performance and are of particular use in more demanding sensing applications, such as ICU monitoring.

It is generally known that in some circumstances, a layer may alter or reduce the sensitivity of some glucose oxidase-based sensor assemblies to competing electroactive species. Accordingly, it is generally believed that a layer is only needed or should be employed on the electroactive surface(s) of the working electrodes where the electrochemical redox reactions of the analyte occurs. However, the Applicants have surprisingly reasoned that a layer can be employed not only on the electroactive surface(s) of the working electrodes, but also on at least one of the non-working electrodes. As a result, the herein disclosed sensor is configured to prevent or substantially eliminate endogenous or exogenous components from contacting or otherwise fouling of the non-working electrode surfaces (e.g., reference electrode surface and/or counter electrode surface).

In one aspect, an electrochemical sensor is provided comprising a layer covering at least a portion of a non-working electrode thereof, where endogenous or exogenous fouling of the non-working electrodes are reduced.

In one aspect, an electrochemical sensor is provided comprising a first layer covering at least a portion of a reference electrode and/or covering at least a portion of a counter electrode thereof.

In one aspect, an electrochemical sensor is provided comprising a first layer in contact with at least a portion of at least one non-working electrode and a second layer disposed on at least a portion of at least one working electrode thereof. In one aspect, the first and second layers are different.

In one aspect, an electrochemical analyte sensor is provided. The sensor comprises a least a portion of the working electrode electroactive surface and covering at least a portion of the reference electrode.

In another aspect, an electrochemical analyte sensor is provided. The sensor comprises a working electroactive surface, a reference electroactive surface, a layer covering at least a portion of the working electroactive surface and covering at least a portion of the reference electrode, and a membrane covering the layer and at least a portion of the electroactive surface.

In one aspect, a method of reducing fouling of a non-working electrode of an electrochemical analyte sensor is provided. The method comprising providing an electrochemical analyte sensor comprising at least one non-working electrode, each of the at least one non-working electrode comprising an electroactive surface; and providing a layer in contact with at least a portion of the electroactive surface of the at least one non-working electrode. The layer reduces fouling of the electroactive surface of the at least one non-working electrode

In another aspect, a method of intravenously measuring an analyte in a subject is provided. The method comprises providing a catheter comprising the sensor assembly as described herein, introducing the catheter into the vascular system of a subject, and measuring an analyte.

### BRIEF DESCRIPTION OF THE DRAWINGS

FIG. 1 shows an amperometric sensor in the form of a flex circuit having a working electrode and a reference electrode deposited with a layer.

FIG. 2 is a side cross-sectional view of a working electrode portion of the sensor.

FIG. 3 is a cross-sectional view of the working electrode portion of the sensor as in FIG. 2.

FIG. 4 is a side view of a multilumen catheter with a sensor assembly.

FIG. 5 is a detail of the distal end of the multilumen catheter of FIG. 4.

### DETAILED DESCRIPTION

While it is believed that the layer on the working electrode (WE) may inhibit diffusion of blood components including interferants, the absence of a anti-fouling layer on the non-working electrodes (reference electrode (RE) or counter electrode (CE)) renders the non-working electrodes vulnerable or susceptible to chemical/physical changes, fouling or alteration by the deposition of blood components or interferants. Thus, following description and examples provide amperometric sensing, in which the concentration of an analyte present in a patient may be determined by a sensor comprising an enzyme electrode sensor that produces a rapid and accurate electrical current proportional to the true analyte concentration, where endogenous or exogenous fouling of the non-working electrodes are reduced or substantially eliminated. The following description and examples illustrate some exemplary embodiments disclosed and described herein. Those of skill in the art will recognize that there may be numerous variations and modifications of that disclosed and described herein that may be encompassed by its scope. Accordingly, the description of a certain exemplary embodiment is not intended to limit the scope of the claims.

### DEFINITIONS

In order to facilitate an understanding of the various aspects disclosed and described herein, the following are defined below.

The term “analyte” as used herein refers without limitation to a substance or chemical constituent of interest in a biological fluid (for example, blood) that may be analyzed. The analyte may be naturally present in the biological fluid, the analyte may be introduced into the body, or the analyte may be a metabolic product of a substance of interest or an enzymatically produced chemical reactant or chemical product of a substance of interest. Preferably, analytes an electrochemically reactive product that is either amperiometrically or voltammetrically detectable.

The phrases and terms “analyte measuring device,” “sensor,” and “sensor assembly” as used herein refer without limitation to an area of an analyte-monitoring device that enables the detection of at least one analyte. For example, the sensor may comprise a non-conductive portion, at least one working electrode, a reference electrode, and at least one counter electrode (optional), forming an electrochemically reactive surface at one location on the non-conductive portion and an electronic connection at another location on the non-conductive portion, and a one or more layers over the electrochemically reactive surface.

The phrase “capable of” as used herein, when referring to recitation of function associated with a recited structure, is inclusive of all conditions where the recited structure can actually perform the recited function. For example, the phrase “capable of” includes performance of the function under normal operating conditions, experimental conditions or laboratory conditions, as well as conditions that may not or can not occur during normal operation.

The phrase “cellulose acetate butyrate” as used herein refers without limitation to compounds obtained by contacting cellulose with acetic anhydride and butyric anhydride.

The term “comprising” and its grammatical equivalents, as used herein is synonymous with “including,” “containing,” or “characterized by,” and is inclusive or open-ended and does not exclude additional, un-recited elements or method steps.

The phrases “continuous analyte sensing” and “continual analyte sensing” (and the grammatical equivalents “continuously” and continually”) as used herein refer without limitation to a period of analyte concentration monitoring that is continuously, continually, and/or intermittently (but regularly) performed.

The phrase “continuous glucose sensing” as used herein refers without limitation to a intermittently (but regularly) performed. The period may, for example, at time intervals ranging from fractions of a second up to, for example, 1, 2, or 5 minutes, or longer.

The phrase “counter electrode” as used herein refers without limitation to an electrode paired with a working electrode, through which passes an electrochemical current equal in magnitude and opposite in sign to the current passed through the working electrode. As used herein, the term “counter electrode” is meant to include counter electrodes which also function as reference electrodes (i.e. a counter/reference electrode).

The terms “crosslink” and “crosslinking” as used herein refer without limitation to joining (e.g., adjacent chains of a polymer and/or protein) by creating covalent or ionic bonds. Crosslinking may be accomplished by known techniques, for example, thermal reaction, chemical reaction or ionizing radiation (for example, electron beam radiation, UV radiation, X-ray, or gamma radiation). For example, reaction of a dialdehyde such as glutaraldehyde with a hydrophilic polymer-enzyme composition would result in chemical crosslinking of the enzyme and/or hydrophilic polymer.

The terms “cover” and “dispose” and their grammatical equivalents is used herein refers without limitation to their respective normal dictionary definitions. The terms cover or dispose are inclusive of one or more intervening layers. For example, a layer covering or disposed on at least a portion of an electroactive surface is inclusive of one or more intervening layers between the layer and the electroactive surface.

The phrase “electroactive surface” as used herein is refers without limitation to a surface of an electrode where an electrochemical reaction takes place. The electroactive surface includes the surface of any of one or more working electrodes (WE), any of one or more reference electrodes (RE), any one or more blank electrodes (BE), and any of one or more counter electrodes (CE). For example, at a predetermined potential, H₂O₂ reacts with the electroactive surface of a working electrode to produce two protons (2H+), two electrons (2e⁻) and one electroactive surface may include on at least a portion thereof, a chemically or covalently bonded adhesion promoting agents such as aminoalkylsilanes and the like.

The terms “interferants,” “interferents” and the phrase “interfering species,” as used herein refer without limitation to effects and/or species that otherwise interfere with a measurement of an analyte of interest in a sensor to produce a signal that does not accurately represent the analyte measurement. For example, in an electrochemical sensor, interfering species may be compounds with oxidation or reduction potentials that substantially overlap the oxidation potential of the analyte to be measured.

The phrase “enzyme layer” as used herein refers without limitation to a permeable or semi-permeable layer comprising an enzyme contained within one or more domains that may be permeable to reactants and/or co-reactants employed in determining the analyte of interest. As an example, an enzyme layer comprises an immobilized glucose oxidase enzyme in a hydrophilic polymer, which catalyzes an electrochemical reaction with glucose and oxygen to permit measurement of a concentration of glucose.

The phrase “non-working electrode” as used herein refers to any electrode of a sensor other than the working electrode, where the working electrode is the electrode where the reaction of interest is occurring. The non-working electrode may be cathodic or anodic. By way of example, for a glucose sensor, a non-working electrode includes a reference electrode, a counter electrode or auxiliary electrode, a blank electrode and combinations thereof. In a preferred aspect, the non-working electrode is cathodic and includes a reference electrode, a counter electrode or auxiliary electrode and combinations thereof.

The term “membrane” as used herein refers to a semi-permeable material that restricts or inhibits the flux of oxygen and other analytes. Preferably, the membrane restricts or inhibits the flux of oxygen and other analytes from accessing the underlying enzyme layer. By way of example, for a glucose sensor, the membrane preferably renders oxygen to an underlying layer in a non-rate-limiting excess. As a result, the upper limit of linearity of glucose measurement is extended to a much higher value than that which is achieved without the membrane.

The term “subject” as used herein refers without limitation to mammals, particularly humans and domesticated animals.

### Sensor System and Sensor Assembly

The aspects herein disclosed and described relate to the use of an analyte sensor system that measures a concentration of analyte of interest or a substance indicative of the concentration or presence of the analyte. The sensor system is a continuous device, and may be used, for example, as or part of a subcutaneous, transdermal (e.g., transcutaneous), or intravascular device. The analyte sensor may use an enzymatic, chemical, electrochemical, or combination of such methods for analyte-sensing. The output signal is typically a raw signal that is used to provide a useful value of the analyte of interest to a user, such as a patient or physician, who may be using the device. Accordingly, appropriate smoothing, calibration, and evaluation methods may be applied to the raw signal.

Generally, the sensor comprises at least a portion of the exposed electroactive surface of a working electrode surrounded by one or more layers. Preferably, a layer is deposited over at least a portion of at least two different electroactive surfaces of the sensor (for example, the working electrode and the reference electrode) to reduce or eliminate changes or alterations of the non-working electrode performance and/or provide protection of the exposed non-working electrode surfaces from the biological environment and/or limit or block of interferants.

Other layers, such as an enzyme layer and a membrane (e.g., flux-limiting layer) may be provided over the electroactive surfaces as described above. Preferably, the enzyme layer is positioned over a layer covering the electroactive surface of the working electrode, such as a hydrophilic polymer. The enzyme layer may be deposited over and may be in direct contact with at least a portion of the layer covering the electroactive surface of the working electrode.

One exemplary embodiment described in detail below utilizes a medical device, such as a catheter, with a glucose sensor assembly. In one aspect, a medical device with an analyte sensor the analyte sensor assembly may include associated therewith an electronics unit associated with the sensor, and a receiver for receiving and/or processing sensor data. Although a few exemplary embodiments of continuous glucose sensors may be illustrated and described herein, it should be understood that the disclosed embodiments may be applicable to any device capable of substantially intermittent or substantially continuous measurement of a signal representative of a concentration of an analyte of interest and/or for providing a rapid and accurate output signal that is representative of the concentration of that analyte.

### Electrode and Electroactive Surface

The electrode and/or the electroactive surface of the sensor or sensor assembly disclosed herein comprises a conductive material, such as platinum, platinum-iridium, palladium, graphite, gold, carbon, conductive polymer, alloys, conductive ink, silver or the like. Although the electrodes can by formed by a variety of manufacturing techniques (bulk metal processing, deposition of metal onto a substrate, or the like), it may be advantageous to form the electrodes from screen printing techniques using conductive and/or catalyzed inks. The conductive inks may be catalyzed with noble metals such as platinum and/or palladium.

The reference electrode and/or the electroactive surface of the reference electrode may be a metal and a salt of the metal in substantial equilibrium with each other, the cation of the metal being capable of participating in a reversible chemical reaction. The anion may be one that forms a substantially insoluble compound with the metal cation. For example, the reference electrode may be of the silver/silver chloride type, the anion is one that forms a substantially insoluble compound with the metal cation is a chloride anion. In one aspect, the reference electrode is of a silver/silver chloride construction.

In one aspect, silver metal is deposited onto the sensor substrate, and subsequently chloridized to form silver/silver chloride reference electrode. Chloridizing the silver metal enables the manufacture of a reference electrode with optimal in vivo performance. For example, by controlling the quantity and amount of chloridization of the silver to form a silver/silver life may result. Additionally, construction of the silver/silver chloride as described above allows for relatively inexpensive and simple manufacture of the reference electrode.

In one aspect, the electrodes and/or the electroactive surfaces of the sensor or sensor assembly are formed on a flexible substrate. In one aspect, the electrodes and/or the electroactive surfaces of the sensor or sensor assembly are formed on a flexible substrate that is a flex circuit. In one aspect, a flex circuit is part of the sensor and comprises a substrate, conductive traces, and electrodes. The traces and electrodes may be masked and imaged onto the substrate, for example, using screen printing or ink deposition techniques. The trace and the electrodes, and the electroactive surface of the electrode may be comprised of a conductive material, such as platinum, platinum-iridium, palladium, graphite, gold, carbon, silver, conductive polymer, alloys, ink or the like.

In one aspect, a counter electrode is provided to balance the current generated by the species being measured at the working electrode. In the case of a glucose oxidase based glucose sensor, the species being measured at the working electrode is H₂O₂. Glucose oxidase catalyzes the conversion of oxygen and glucose to hydrogen peroxide and gluconate according to the following reaction: Glucose+O₂→Gluconate+H₂O₂. Oxidation of H₂O₂ by the working electrode is balanced by reduction of any oxygen present, or other reducible species at the counter electrode. The H₂O₂ produced from the glucose oxidase reaction reacts at the surface of working electrode and produces two protons (2H⁺), two electrons (2e⁻), and one oxygen molecule (O₂). In one aspect, the electroactive surface of the counter electrode and the working electrode are covered by a layer as described herein. In another aspect, the electroactive surface of the counter electrode, the reference electrode and the working electrode are covered by a layer as described herein.

In one aspect, additional electrodes may be included within the sensor or sensor assembly, for example, a three-electrode system (working, reference, blank and counter electrodes) and/or one or more additional working electrodes configured as a baseline subtracting electrode, or which is configured for measuring additional analytes. The two working electrodes electrode. For example, a multiple electrode system may be configured wherein a first working electrode is configured to measure a first signal comprising glucose and baseline and an additional working electrode substantially similar to the first working electrode without an enzyme disposed thereon is configured to measure a baseline signal consisting of baseline only. In this way, the baseline signal generated by the additional electrode may be subtracted from the signal of the first working electrode to produce a glucose-only signal substantially free of baseline fluctuations and/or electrochemically active interfering species.

In one aspect, the sensor comprises from 2 to 4 electrodes. The electrodes may include, for example, the counter electrode (CE), working electrode (WE), reference electrode (RE) and optionally a second working electrode, or blank electrode (BE). In one aspect, the sensor will have at least a CE and WE. In one aspect, the addition of a BE is used, which may further improve the accuracy of the sensor measurement.

The electroactive surface of the electrodes (WE, CE, BE and RE) may be treated prior to application of any subsequent layers, including the layer described herein. Surface treatments may include for example, chemical, plasma or laser treatment of at least a portion of the electroactive surface. The electrodes may be chemically or covalently contacted with one or more adhesion promoting agents. Adhesion promoting agents may include for example, aminoalkylalkoxylsilanes, epoxyalkylalkoxylsilanes and the like. For examples, one or more of the electrodes may be chemically or covalently contacted with a solution containing 3-glycidoxypropyltrimethoxysilane.

In some alternative embodiments, the exposed surface area of the working (and/or other) electrode may be increased by altering the cross-section of the electrode itself. Increasing the surface area of the working electrode may be advantageous in providing an increased signal responsive to the analyte concentration, which in turn may be helpful in improving the signal-to-noise ratio, for example. The cross-section of the working electrode may be defined by any regular or irregular, circular or non-circular configuration.

In some applications, cellular attack or migration of cells to the sensor can cause reduced sensitivity and/or function of the device, particularly after the first day of implantation. However, when the exposed electroactive surface of the non-working electrode is coated with a layer as herein described, reduction or elimination of local cellular contact and/or deposition of the electroactive surface is envisaged. Other methods and configurations for preventing cellular contact of the exposed electroactive surface of the non-working electrode may be used in combination with the methods disclosed herein.

### Non-working Electrode Layer

The electroactive surface of the non-working electrode may be coated with a layer capable of eliminating or reducing fouling. For example, the electroactive surface of the non-working electrode may be coated with a material selected from cellulose ester derivatives, silicones, polytetrafluoroethylenes, polyethylene-co-tetrafluoroethylenes, polyolefins, polyesters, polycarbonates, biostable polytetrafluoroethylenes, homopolymers, copolymers, terpolymers of polyurethanes, polypropylenes (PP), polyvinylchloride (PVC), polyvinylidene fluoride (PVDF), polybutylene terephthalates (PBT), polymethylmethacrylates (PMMA), polyether ether ketones (PEEK), polyurethanes, cellulosic polymers, polysulfones, tetrafluoroethylene-perfluoro-3,6-dioxa-4-methyl-7-octenesulfonic acid copolymer (Nafion) and block copolymers thereof including, for example, di-block, tri-block, alternating, random and graft copolymers. Combinations of the above polymers may be used. In one preferred aspect, the layer is an interferant layer, such that the layer is effective at reducing or eliminating diffusion of interfering species relative to, for example, hydrogen peroxide.

In one aspect, the layer is formed from one or more cellulosic derivatives. Cellulosic derivatives can include, but are not limited to, cellulose esters and cellulose ethers. In general, cellulosic derivatives include polymers such as cellulose acetate, cellulose acetate butyrate, 2-hydroxyethyl cellulose, cellulose acetate phthalate, cellulose acetate propionate, cellulose acetate trimellitate, and the like, as well as their copolymers and terpolymers with other cellulosic or non-cellulosic monomers. While cellulosic derivatives are generally preferred, other polymeric polysaccharides having similar properties to cellulosic derivatives may also be employed.

In one aspect, the layer deposited on the electroactive surface of the at least one non-working electrode is formed from cellulose acetate butyrate. Cellulose acetate butyrate is a cellulosic polymer having both acetyl and butyl groups, and may also include hydroxyl groups. A cellulose acetate butyrate having about 35% or less acetyl groups, about 10% to about 25% butyryl groups, and hydroxyl groups making up the remainder may be used. A cellulose acetate butyrate having from about 25% to about 34% acetyl groups and from about 15 to about 20% butyryl groups may also be used, however, other amounts of acetyl and butyryl groups may be used. A preferred cellulose acetate butyrate contains from about 28% to about 30% acetyl groups and from about 16 to about 18% butyryl groups.

Cellulose acetate butyrate with a molecular weight of about 10,000 daltons to about 75,000 daltons is preferred, preferably from about 15,000, 20,000, or 25,000 daltons to about 50,000, 55,000, 60,000, 65,000, or 70,000 daltons, and more preferably about 65,000 daltons is employed. In certain embodiments, however, higher or lower molecular weights may be used or a blend of two or more cellulose acetate butyrates having different molecular weights may be used.

A plurality of layers of cellulose acetate butyrate may be combined to form the layer in some embodiments, for example, two or more layers may be employed. It may be desirable to employ a mixture of cellulose acetate butyrates with different molecular weights in a single solution, or to deposit multiple layers of cellulose acetate butyrate from different solutions comprising cellulose acetate butyrate of different molecular weights, different concentrations, and/or different chemistries (e.g., wt % functional groups). Additional substances in the casting solutions or dispersions may be used, e.g., casting aids, defoamers, surface tension modifiers, functionalizing agents, crosslinking agents, other polymeric substances, substances capable of modifying the hydrophilicity/hydrophobicity of the resulting layer, and the like.

The precursor composition of the layer may be sprayed, cast, deposited, or dipped directly to the electroactive surface(s) of the at least one non-working electrode. The dispensing of the precursor composition of the layer may be performed using any known thin film technique. Two, three or more layers of precursor composition of the layer may be formed by the sequential application and curing and/or drying.

The concentration of solids in the precursor composition of the layer may be adjusted to deposit a sufficient amount of solids or film on the electrode in one layer (e.g., in one dip or spray) to form a layer sufficient to eliminate or reduce fouling of the electroactive surface of the non-working electrode. For example, the precursor composition's percentage of solids may be adjusted such that only a single layer is required to deposit a sufficient amount to form a functional layer. A sufficient amount of precursor composition would be an amount that provides a layer that substantially eliminates or reduces fouling of the electroactive surface of the non-working electrode.

In one aspect, the layer is deposited either directly onto the electroactive surface of the non-working electrode or onto a material or layer in direct contact therewith. Preferably, the layer is deposited directly onto at least a portion of the electroactive surface of the non-working electrode substantially without an intervening material or layer in direct contact therewith. Substantially without an intervening material or layer in direct contact with the non-working electrode would allow for the presence of minor amounts of adhesion promoting materials and naturally occurring oxidation layers on the electroactive surface of the electrode.

The layer may be applied to the electroactive surface of the non-working electrode provide a thickness of from about 0.05 micron or less to about 20 microns or more, more preferably from about 0.05, 0.1, 0.15, 0.2, 0.25, 0.3, 0.35, 0.4, 0.45, 0.5, 1, 1.5, 2, 2.5, 3, or 3.5 microns to about 4, 5, 6, 7, 8, 9, 10, 11, 12, 13, 14, 15, 16, 17, 18, 19, or 19.5 microns, and more preferably still from about 1, 1.5 or 2 microns to about 2.5 or 3 microns. Thicker membranes may also be desirable in certain embodiments, but thinner membranes may be generally preferred.

In one aspect, polymers, such as Nafion®, may be used alone or in combination with a cellulosic derivative to provide the layer for the electroactive surface of the non-working electrode. For example, a layer of a 5 wt. % Nafion® casting solution was applied over a previously applied (e.g., and cured) layer of 8 wt. % cellulose acetate, e.g., by dip coating at least one layer of cellulose acetate and subsequently dip coating at least one layer Nafion® onto the electroactive surface of the non-working electrode. Any number of coatings or layers formed in any order may be suitable for forming the layer on the electroactive surface of the non-working electrode.

In other aspects, other polymer types may be utilized as a base material for the layer on the electroactive surface of the non-working electrode. For example, polyurethanes, polymers having pendant ionic groups, and polymers having controlled pore size, for example. By way of example, the layer on the non-working electrode may include a thin, hydrophobic membrane that is substantially non-swellable and restricts diffusion of high molecular weight species, such as biological components.

### Enzyme Layer

The sensor or sensor assembly disclosed herein generally includes an enzyme layer comprising an enzyme composition.

In one aspect, the enzyme layer comprises a enzyme and a hydrophilic polymer. The hydrophilic polymer may be selected from poly-N-vinylpyrrolidone, poly-N-vinyl-3-ethyl-2-pyrrolidone, poly-N-vinyl-4,5-dimethyl-2-pyrrolidone, polyacrylamide, poly-N,N-dimethylacrylamide, polyvinyl alcohol, polymers with pendent ionizable groups and copolymers thereof. Preferably, the enzyme layer comprises poly-N-vinylpyrrolidone. In one aspect, the enzyme layer comprises glucose oxidase, poly-N-vinylpyrrolidone and an amount of crosslinking agent sufficient to immobilize the enzyme and/or the poly-N-vinylpyrrolidone.

Most importantly, the molecular weight of the hydrophilic polymer of the enzyme layer is such that fugitive species are prevented or substantially inhibited from leaving the sensor environment and more particularly, fugitive species are prevented or substantially inhibited from leaving the enzyme's environment when the sensor is initially put into use.

The hydrophilic polymer-enzyme composition of the enzyme layer may further include at least one protein and/or natural or synthetic material. For example, the hydrophilic polymer-enzyme composition of the enzyme layer may further include, for example, serum albumins, polyallylamines, polyamines and the like, as well as combination thereof.

In one aspect, other proteins or natural or synthetic materials may be substantially excluded from the hydrophilic polymer-enzyme composition of the enzyme layer. For example, the hydrophilic polymer-enzyme composition may be substantially free of bovine serum albumin. Bovine albumin-free compositions may be desirable for meeting various governmental regulatory requirements. Thus, in one aspect, the hydrophilic polymer-enzyme composition of the enzyme layer consists essentially of glucose oxidase, poly-N-vinylpyrrolidone and a cross-linking agent, for example, a dialdehyde such as glutaraldehyde, to cross-link or otherwise immobilize the components of the composition.

In one aspect, the enzyme composition comprises glucose oxidase, bovine serum albumin, and poly-N-vinylpyrrolidone. The composition may further include a cross-linking agent, for example, a dialdehyde such as glutaraldehyde, to cross-link or otherwise immobilize the components of the composition. In one aspect, the enzyme is encapsulated within a hydrophilic polymer and may be cross-linked or otherwise immobilized therein.

The enzyme layer thickness may be from about 0.05 microns or less to about 20 microns or more, more preferably from about 0.05, 0.1, 0.15, 0.2, 0.25, 0.3, 0.35, 0.4, 0.45, 0.5, 1, 1.5, 2, 2.5, 3, or 3.5 microns to about 4, 5, 6, 7, 8, 9, 10, 11, 12, 13, 14, 15, 16, 17, 18, 19, or 19.5 microns. Preferably, the enzyme domain is deposited by spray or dip coating, however, other methods of forming the enzyme layer may be used. The enzyme layer may be formed by dip coating and/or spray coating one or more layers at a predetermined concentration of the coating solution, insertion rate, dwell time, withdrawal rate, to provide the desired thickness.

### Membrane Layer

The sensor or sensor assembly may further include a membrane disposed over the subsequent layers described above. The membrane may function as a flux limiting membrane. Although the following description is directed to a flux limiting membrane for a glucose sensor, the flux limiting membrane may be modified for other analytes and co-reactants as well. In one aspect, the sensor or sensor assembly includes a flux limiting membrane disposed on the layer as herein disclosed.

The flux limiting membrane comprises a semipermeable material that controls the flux of oxygen and glucose to the underlying enzyme layer, preferably providing oxygen in a non-rate-limiting excess. As a result, the upper limit of linearity of glucose measurement is extended to a much higher value than that which is achieved without the flux limiting membrane. In one embodiment, the flux limiting membrane exhibits an oxygen to glucose permeability ratio of from about 50:1 or less to about 400:1 or more, preferably about 200:1.

In one aspect, the material that comprises the flux limiting membrane may be a vinyl polymer appropriate for use in sensor devices as having sufficient permeability to allow relevant compounds to pass through it, for example, to allow an oxygen molecule to pass through in order to reach the active enzyme or electrochemical electrodes. Examples of materials which may be used to make flux limiting membranes include vinyl polymers having vinyl acetate monomeric units. In a preferred embodiment, the flux limiting membrane comprises poly ethylene vinylacetate (EVA polymer) having a vinyl acetate content of at least 33 wt. %. In other aspects, the flux limiting membrane comprises poly(methylmethacrylate-co-butyl methacrylate) blended with the EVA polymer having a vinyl acetate content of at least 33 wt. %. The EVA polymer or its blends may be cross-linked, for example, with diglycidyl ether.

In one aspect, the flux limiting membrane excludes condensation polymers such as silicone and urethane polymers and/or copolymers or blends thereof. Such excluded condensation polymers typically contain residual heavy metal catalytic material that may otherwise be toxic if leached and/or difficult to completely remove, thus rendering their use in such sensors undesirable for safety and/or cost.

The EVA polymer may be provided from a source having a composition anywhere from greater than 33 wt. % vinyl acetate (EVA-33) to more than 40 wt. % vinyl acetate (EVA-40). The EVA polymer is preferably dissolved in a solvent for dispensing on the sensor or sensor assembly. The solvent should be chosen for its ability to dissolve EVA polymer, to promote adhesion to the sensor substrate and enzyme electrode, and to form a solution that may be effectively applied (e.g. spray-deposited or dip deposited). Solvents such as cyclohexanone, paraxylene, and tetrahydrofuran may be suitable for this purpose. The solution may include about 0.5 wt % to about 10.0 wt % of the EVA polymer. In addition, the solvent should be sufficiently volatile to evaporate without undue agitation to prevent issues with the underlying enzyme, but not so volatile as to create problems with the spray process. In a preferred embodiment, the vinyl acetate component of the flux limiting membrane includes at least 33 wt. % vinyl acetate. In preferred embodiments, the flux limiting membrane is deposited onto the enzyme domain to yield a domain thickness of from about 0.05 microns or less to about 20 microns or more, more preferably from about 0.05, 0.1, 0.15, 0.2, 0.25, 0.3, 0.35, 0.4, 0.45, 0.5, 1, 1.5, 2, 2.5, 3, or 3.5 microns to about 4, 5, 6, 7, 8, 9, 10, 11, 12, 13, 14, 15, 16, 17, 18, 19, or 19.5 microns, and more preferably still from about 5, 5.5 or 6 microns to about 6.5, 7, 7.5 or 8 microns. The flux limiting membrane may be deposited onto the enzyme domain by spray coating or dip coating. In one aspect, the flux limiting membrane is deposited on the enzyme domain by dip coating a solution of from about 1 wt. % to about 10 wt. % EVA polymer and from about 95 wt. % to about 99 wt. % solvent.

Other flux limiting membranes may be used or combined, such as a membrane with both hydrophilic and hydrophobic regions to control the diffusion of analyte and optionally co-analyte to an analyte sensor. For example, a suitable membrane may include a hydrophobic polymer component such as a polyurethane, or polyetherurethaneurea.

The material that forms the basis of the hydrophobic matrix of the membrane can be any of those known in the art as appropriate for use as membranes in sensor devices and as having sufficient permeability to allow relevant compounds to pass through it, for example, to allow an oxygen molecule to pass through the membrane from the sample under examination in order to reach the active enzyme or electrochemical electrodes. For example, non-polyurethane type membranes such as vinyl polymers, polyethers, polyesters, polyamides, inorganic polymers such as polysiloxanes and polycarbosiloxanes, natural polymers such as cellulosic and protein based materials, and mixtures or combinations thereof may be used.

In one aspect, the flux limiting layer comprises polyethylene oxide. For example, a hydrophobic-hydrophilic copolymer comprising polyethylene oxide is a polyurethane polymer that includes about 20% hydrophilic polyethylene oxide. The polyethylene oxide portions of the copolymer are thermodynamically driven to separate from the hydrophobic portions (e.g., the urethane portions) of the copolymer and the hydrophobic polymer component. The 20% polyethylene oxide-based soft segment portion of the copolymer used to form the final blend affects the water pick-up and subsequent glucose permeability of the membrane.

In one aspect, an electrochemical analyte sensor is provided comprising a membrane covering at least a portion of the electroactive surfaces having deposited thereon the layers as disclosed above. Thus, in one aspect, the sensor comprises at working electroactive surface, a reference electroactive surface, a first layer contacting at least a portion of the working electroactive surface and a second layer contacting at least a portion of a non-working electrode, and a membrane covering the first and second layers.

### Bioactive Agents

In some alternative embodiments, a bioactive agent may be optionally incorporated into the above described sensor system, such that the bioactive diffuses out into the biological environment adjacent to at least one of the sensor components. For example, bioactive agents may be selected from anti-inflammatory agents, anti-fouling agents, anti-platelet agents, anti-coagulants, anti-proliferates, cytotoxic agents, anti-barrier cell compounds, or mixtures thereof.

### Flexible Substrate/Flex Circuit Sensor Assembly Adapted for Intravenous Insertion

In one aspect, an electrochemical analyte sensor assembly may be configured for an intravenous insertion to a vascular system of a subject. In order to accommodate the sensor within the confined space of a device suitable for intravenous insertion, the sensor assembly may comprise a flexible substrate. Preferably the flexible substrate is a flex circuit. The flex circuit may comprise at least one non-working electrode and at least one working electrode, the at least one working electrode having an electroactive surface capable of providing a detectable electrical output upon interaction with an electrochemically detectable species. A first layer may be placed in direct contact with a portion of the electroactive surface of the at least one non-working electrode and a second layer may be placed in direct contact with a portion of the electroactive surface of the one or more working electrodes. An enzyme layer comprising a hydrophilic polymer-enzyme composition capable of enzymatically interacting with an analyte so as to provide the electrochemically detectable species, may be placed in direct contact with and at least partially covering the second layer covering the working electrode. And a membrane may be placed such that it covers the hydrophilic polymeric layer, the first and second layers and at least a portion of the electroactive surfaces of the working and non-working electrodes. The flex circuit preferably is configured to electrically couple to a control unit. An example of a flex circuit and it construction is found in co-assigned U.S. Application Nos. 2007/0202672 and 2007/0200254, incorporated herein by reference in their entirety.

Medical devices adaptable to the sensor assembly as described above include, but are not limited to a central venous catheter (CVC), a pulmonary artery catheter (PAC), a probe for insertion through a CVC or PAC or through a peripheral IV catheter, a peripherally inserted catheter (PICC), Swan-Ganz catheter, an introducer or an attachment to a Venous Arterial blood Management Protection (VAMP) system. Any size/type of Central Venous Catheter (CVC) or intravenous devices may be used or adapted for use with the sensor assembly.

For the foregoing discussion, the implementation of the sensor or sensor assembly is disclosed as being placed within a catheter, however, other devices as described above are envisaged and incorporated in aspects disclosed and described. The sensor assembly will preferably be applied to the catheter so as to be flush with the OD of the catheter tubing. This may be accomplished, for example, by thermally deforming the OD of the tubing to provide a recess for the sensor. The sensor assembly may be bonded in place, and sealed with an adhesive (i.e. urethane, 2-part epoxy, acrylic, etc.) that will resist bending/peeling, and adhere to the urethane CVC tubing, as well as the materials of the sensor. Small diameter electrical wires may be attached to the sensor assembly by soldering, resistance welding, or conductive epoxy. These wires may travel from the proximal end of the sensor, through one of the catheter lumens, and then to the proximal end of the catheter. At this point, the wires may be soldered to an electrical connector.

The sensor assembly as disclosed herein can be added to a catheter in a variety of ways. For example, an opening may be provided in the catheter body and a sensor or sensor assembly may be mounted inside the lumen at the opening so that the sensor would have direct blood contact. In one aspect, the sensor or sensor assembly may be positioned proximal to all the infusion ports of the catheter. In this configuration, the sensor would be prevented from or minimized in measuring otherwise detectable infusate concentration instead of the blood concentration of the analyte. Another aspect, an attachment method may be an indentation on the outside of the catheter body and to secure the sensor inside the indentation. This may have the added advantage of partially isolating the sensor from the temperature effects of any added infusate. Each end of the recess may have a skived opening to 1) secure the distal end of the sensor and 2) allow the lumen to carry the sensor wires to the connector at the proximal end of the catheter.

Preferably, the location of the sensor assembly in the catheter will be proximal (upstream) of any infusion ports to prevent or minimize IV solutions from affecting analyte measurements. In one aspect, the sensor assembly may be about 2.0 mm or more proximal to any of the infusion ports of the catheter.

In another aspect, the sensor assembly may be configured such that flushing of the catheter (i.e. saline solution) may be employed in order to allow the sensor assembly to be cleared of any material that may interfere with its function.

### Sterilization of the Sensor or Sensor Assembly

Generally, the sensor or the sensor assembly as well as the device that the sensor is adapted to are sterilized before use in a subject. Sterilization may be achieved using radiation (e.g., electron beam or gamma radiation), ethylene oxide or flash-UV sterilization, or other means know in the art.

Disposable portions, if any, of the sensor, sensor assembly or devices adapted to receive the and contain the sensor preferably will be sterilized, for example using e-beam or gamma radiation or other know methods. The fully assembled device or any of the disposable components may be packaged inside a sealed non-breathable container or pouch.

Referring now to the Figures, FIG. 1 is an amperometric sensor 11 in the form of a flex circuit that incorporates a sensor embodiment disclosed and described. The sensor or sensor 11 may be formed on a flexible substrate 13 (e.g., a flex circuit, such as copper foil laminated with polyimide). One or more electrodes 15, 17 and 19 may be attached or bonded to a surface of the substrate 13. The sensor 11 is shown with a reference electrode 19, a counter electrode 17, and a working electrode 15. In another embodiment, one or more additional working electrodes may be included on the substrate 13. Electrical wires 210 may transmit power to the electrodes for sustaining an oxidation or reduction reaction, and may also carry signal currents to a detection circuit (not shown) indicative of a parameter being measured. The parameter being measured may be any analyte of interest that occurs in, or may be derived from, blood chemistry. In one embodiment, the analyte of interest may be hydrogen peroxide, formed from reaction of glucose with glucose oxidase, thus having a concentration that is proportional to blood glucose concentration.

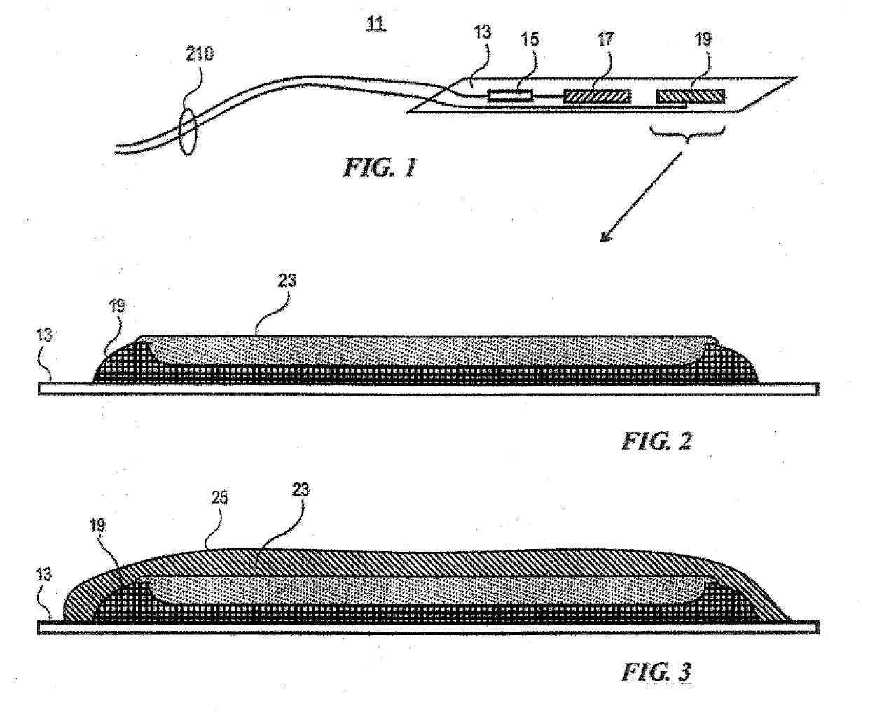

FIG. 2 depicts a cross-sectional side view of a portion of substrate 13 in the vicinity of a non-working electrode (e.g., reference electrode 19). The non-working electrode may be at least partially deposited with a layer 23, such as an interference layer.

FIG. 3 shows a cross sectional side view of the non-working electrode site of FIG. 2 on the sensor substrate 13 further comprising membrane 25 covering layer 23 and at least a portion of electrode 19. Membrane 25 may selectively allow diffusion, from blood to an enzyme layer on the working electrode (not shown), a blood component that reacts with the enzyme. In a glucose sensor embodiment, the flux limiting membrane 25 passes an abundance of oxygen, and selectively limits glucose flux. In addition, membrane 25 that has adhesive properties may mechanically seal layer 23 to the electrode and/or substrate, and may also seal or secure the non-working electrode 19 to the sensor substrate 13. It is herein disclosed that a membrane formed from an EVA polymer having a vinyl acetate content of at least 33% may serve as a flux limiter at the top of the electrode, but also serve as a sealant or encapsulant at the enzyme/electrode boundary and at the electrode/substrate boundary. An additional biocompatible layer (not shown), including a biocompatible anti-thrombotic substance such as heparin, may be added onto the membrane 25.

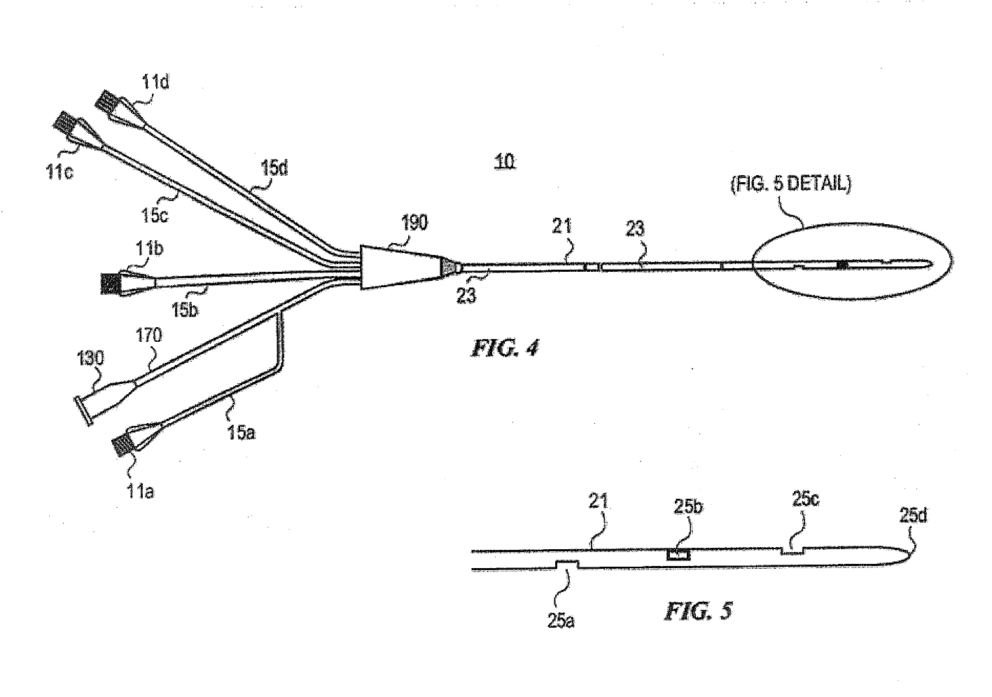

Referring now to FIGS. 4-5, aspects of the sensor adapted to a central line catheter with a sensor or sensor assembly are discussed as exemplary embodiments, without limitation to any particular intravenous device. FIG. 4 shows a sensor assembly within a multilumen catheter. The catheter assembly 10 may include multiple infusion ports 11a, 11b, 11c, 11d and one or more electrical connectors 130 at its most proximal end. A lumen 15a, 15b, 15c or 15d may connect each infusion port 11a, 11b, 11c, or 11d, respectively, to a junction 190. Similarly, the conduit 170 may connect an electrical connector 130 to the junction 190, and may terminate at junction 190, or at one of the lumens 15a-15d (as shown). Although the particular embodiment shown in FIG. 4 is a multilumen catheter having four lumens and one electrical connector, other embodiments having other combinations of lumens and connectors are possible, including a single lumen catheter, a catheter having multiple electrical connectors, etc. In another embodiment, one of the lumens and the electrical connector may be reserved for a probe or other sensor mounting device, or one of the lumens may be open at its proximal end and designated for insertion of the probe or sensor mounting device.

The distal end of the catheter assembly 10 is shown in greater detail in FIG. 5. At one or more intermediate locations along the distal end, the tube 21 may define one or more ports formed through its outer wall. These may include the intermediate ports 25a, 25b, and 25c, and an end port 25d that may be formed at the distal tip of tube 21. Each port 25a-25d may correspond respectively to one of the lumens 15a-15d. That is, each lumen may define an independent channel extending from one of the infusion ports 11a-11d to one of the tube ports 25a-25d. The sensor assembly may be presented to the sensing environment via positioning at one or more of the ports to provide contact with the medium to be analyzed.

Central line catheters may be known in the art and typically used in the Intensive Care Unit (ICU)/Emergency Room of a hospital to deliver medications through one or more lumens of the catheter to the patient (different lumens for different medications). A central line catheter is typically connected to an infusion device (e.g. infusion pump, IV drip, or syringe port) on one end and the other end inserted in one of the main arteries or veins near the patient's heart to deliver the medications. The infusion device delivers medications, such as, but not limited to, saline, drugs, vitamins, medication, proteins, peptides, insulin, neural transmitters, or the like, as needed to the patient. In alternative embodiments, the central line catheter may be used in any body space or vessel such as intraperitoneal areas, lymph glands, the subcutaneous, the lungs, the digestive tract, or the like and may determine the analyte or therapy in body fluids other than blood. The central line catheter may be a double lumen catheter. In one aspect, an analyte sensor is built into one lumen of a central line catheter and is used for determining characteristic levels in the blood and/or bodily fluids of the user. However, it will be recognized that further embodiments may be used to determine the levels of other agents, characteristics or compositions, such as hormones, cholesterol, medications, concentrations, viral loads (e.g., HIV), or the like. Therefore, although aspects may be primarily described in the context of glucose sensors used in the treatment of diabetes/diabetic symptoms, the aspects disclosed and described may be applicable to a wide variety of patient treatment programs where a physiological characteristic is monitored in an ICU, including but not limited to blood gases, pH, temperature and other analytes of interest in the vascular system.

In another aspect, a method of intravenously measuring an analyte in a subject is provided. The method comprises providing a catheter comprising the sensor assembly as described herein and introducing the catheter into the vascular system of a subject. The method further comprises measuring an analyte.

Applicants believe that an electrochemical sensor comprising a working electroactive surface, a non-working electroactive surface, a layer covering at least a portion of the working electroactive surface and covering at least a portion of a non-working electrode prevents or substantially eliminates endogenous or exogenous components from contacting the non-working electrode surface of an electrochemical sensor.

Applicants believe that an electrochemical sensor comprising a working electroactive surface, a reference electroactive surface, a layer covering at least a portion of the working electroactive surface and covering at least a portion of the non-working electrode, and a flux limiting membrane covering the layer and at least a portion of the electroactive surface prevents or substantially eliminates endogenous or exogenous components from contacting the non-working electrode surface of the electrochemical sensor. Applicants believe the disclosure herein may be extrapolated to in vivo applications without undue experimentation by one of ordinary skill in the art.

Accordingly, electrochemical sensors and methods have been provided for measuring an analyte in a subject, including a sensor assembly configured for adaption to a continuous glucose monitoring device or a catheter for insertion into a subject's vascular system having electronics unit electrically coupled to the sensor assembly.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

All numbers expressing quantities of ingredients, reaction conditions, and so forth used in the specification may be to be understood as being modified in all instances by the term “about.” Accordingly, unless indicated to the contrary, the numerical parameters set forth herein may be approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

The above description discloses several methods and materials disclosed and described. This disclosure is susceptible to modifications in the methods and materials, as well as alterations in the fabrication methods and equipment. Such modifications will become apparent to those skilled in the art from a consideration of this disclosure or practice of the aspects disclosed herein. Consequently, it is not intended that this disclosure be limited to the specific embodiments disclosed herein, but that it cover all modifications and alternatives coming within the true scope and spirit of that disclosed and described.

What is claimed is:

1. An electrochemical analyte sensor comprising:
at least one working electrode having an electroactive surface;
at least one non-working electrode having an electroactive surface;
a first layer contacting at least a portion of the electroactive surface of the at least one non-working electrode, the first layer reducing endogenous or exogenous components from contacting the non-working electrode surface; and
a second layer disposed on at least a portion of the electroactive surface of the at least one working electrode.

2. An electrochemical sensor of claim 1, wherein at least one non-working electrode is a counter electrode or a reference electrode.

3. An electrochemical sensor of claim 1, wherein the first layer is selected from the group consisting of cellulosic polymer, polysilicones, polytetrafluoroethylenes, polyethylene-co-tetrafluoroethylenes, polyolefins, polyester, polycarbonates, polyurethanes, polypropylene (PP), polyvinylchloride (PVC), polyvinylidene fluoride (PVDF), polybutylene terephthalates (PBT), polymethylmethacrylates (PMMA), polyether ether ketones (PEEK), polyurethanes, polysulfones, tetrafluoroethylene-perfluoro-3,6-dioxa-4-methyl-7-octenesulfonic acid copolymers (Nafion) and combinations thereof.

4. An electrochemical sensor of claim 1, wherein the first layer is a cellulosic polymer.

5. An electrochemical sensor of claim 4, wherein the cellulosic polymer is cellulose acetate butyrate.

6. An electrochemical sensor of claim 5, wherein the cellulose acetate butyrate comprises:
less than about 35% acetyl groups and less than about 25% butyryl groups; or
from about 25% to about 34% acetyl groups and from about 15% to about 20% butyryl groups; or
about 5% acetyl groups and about 17% butyryl groups.

7. An electrochemical sensor of claim 1, wherein the second layer is a hydrophilic polymer contacting the electroactive surface of the working electrode.

8. An electrochemical sensor of claim 7, further comprising an analyte-responsive enzyme layer disposed on at least a portion of the hydrophilic polymer.

9. An electrochemical sensor of claim 7, wherein the hydrophilic polymer comprises a material selected from the group consisting of poly-N-vinylpyrrolidone, poly-N-vinyl-3-ethyl-2-pyrrolidone, poly-N-vinyl-4,5-dimethyl-2-pyrrolidone, polyvinylimidazole, poly-N—N-dimethylacrylamide, polyacrylamide, polyvinyl alcohol, polyethylene glycol and copolymers thereof.

10. An electrochemical sensor of claim 8, wherein the analyte-responsive enzyme layer comprises glucose oxidase and at least one member selected from the group consisting of poly-N-vinylpyrrolidone, a serum albumin and a crosslinking agent.

11. An electrochemical sensor of claim 1, further comprising an analyte flux limiting membrane covering the second layer, and optionally the first layer.

12. An electrochemical sensor of claim 11, wherein the analyte flux limiting membrane comprises ethylene vinyl acetate having a vinyl acetate content of at least 33 wt. %.

13. An electrochemical sensor of claim 11, wherein the analyte flux limiting membrane comprises ethylene vinyl acetate having a vinyl acetate content of about 40 wt. % vinyl acetate.

14. An electrochemical analyte sensor comprising:
at least one working electrode having an electroactive surface having a hydrophilic polymer contacting at least a portion the of the electroactive surface of the at least one working electrode, and an enzyme layer disposed on or combined with the hydrophilic polymer, wherein the enzyme layer comprises glucose oxidase and poly-N-vinylpyrrolidone;
at least one reference or counter electrode having an electroactive surface and a first layer contacting at least a portion of the electroactive surface of the at least one reference or counter electrode, wherein the first layer is selected from the group consisting of cellulosic polymer, polysilicones, polytetrafluoroethylenes, polyethylene-co-tetrafluoroethylenes, polyolefins, polyester, polycarbonates, polyurethanes, polypropylene (PP), polyvinylchloride (PVC), polyvinylidene fluoride (PVDF), polybutylene terephthalates (PBT), polymethylmethacrylates (PMMA), polyether ether ketones (PEEK), polyurethanes, polysulfones, tetrafluoroethylene-perfluoro-3,6-dioxa-4-methyl-7-octenesulfonic acid copolymers (Nafion) and combinations thereof; and
an analyte flux limiting membrane disposed on the at least one working electrode, and optionally the first layer, wherein the analyte flux limiting membrane comprises ethylene vinyl acetate having a vinyl acetate content of at least 33 wt. %.

15. A method of reducing fouling of a non-working electrode of an electrochemical analyte sensor, the method comprising:
providing an electrochemical analyte sensor comprising at least one non-working electrode, each of the at least one non-working electrode comprising an electroactive surface; and
providing a first layer in contact with at least a portion of the electroactive surface of the at least one non-working electrode;
wherein the layer reduces fouling of the electroactive surface of the at least one non-working electrode.

16. A method of claim 15, wherein the at least one non-working electrode is a counter electrode or a reference electrode.

17. A method of claim 15, wherein the first layer is selected from selected from the group consisting of cellulosic polymer, polysilicones, polytetrafluoroethylenes, polyethylene-co-tetrafluoroethylenes, polyolefins, polyester, polycarbonates, polyurethanes, polypropylene (PP), polyvinylchloride (PVC), polyvinylidene fluoride (PVDF), polybutylene terephthalates (PBT), polymethylmethacrylates (PMMA), polyether ether ketones (PEEK), polyurethanes, polysulfones, tetrafluoroethylene-perfluoro-3,6-dioxa-4-methyl-7-octenesulfonic acid copolymers (Nafion) and combinations thereof.

18. A method of claim 17, wherein the cellulosic polymer is cellulose acetate butyrate.

19. A method of claim 15, further comprising at least one working electrode and an analyte-responsive enzyme layer disposed on at least a portion of the electroactive surface of the at least one working electrode.

20. A method of claim 19, wherein the analyte-responsive enzyme layer comprises glucose oxidase and a hydrophilic polymer.

21. A method of claim 19, wherein the analyte-responsive enzyme layer comprises glucose oxidase and at least one member selected from the group consisting of poly-N-vinylpyrrolidone, a serum albumin and a crosslinking agent.

22. A method of claim 19, further providing an analyte flux limiting membrane covering the at least one working electrode, wherein the membrane comprises ethylene vinyl acetate having a vinyl acetate content of at least 33 wt. %.

23. A method of claim 19, further providing an analyte flux limiting membrane covering the at least one working electrode, wherein the membrane comprises ethylene vinyl acetate having a vinyl acetate content of about 40 wt. %.
